(19) United States
(12) Patent Application Publication
(10) Pub. No.: US 2004/0186358 A1
(43) Pub. Date: Sep. 23, 2004

(54) Monitoring system containing a hospital bed with integrated display

(76) Inventors: Bart Chernow, Fort Lauderdale, FL (US); Michael Salem, Fort Lauderdale, FL (US)

Correspondence Address: BANNER & WITCOFF, LTD., TEN SOUTH WACKER DRIVE, SUITE 3000, CHICAGO, IL, 60606, US

(21) Appl. No.: 10/668,658

(22) Filed: Sep. 23, 2003

Related U.S. Application Data
(60) Provisional application No. 60/413,392, filed on Sep. 25, 2002.

(51) Int. Cl. A61B 5/00

(52) U.S. Cl. 600/300; 128/903

(57) ABSTRACT

A system for monitoring physiological signals from a patient to provide increased acuity flexibility, the system including a hospital bed with an integrated display for displaying physiological data and medical records from a patient and, more particularly, a hospital bed with an integrated display for receiving and displaying a patient's physiological data, such as EKG signals, blood pressure data, temperature readings, pulse, respiration rate data, and pulse oximeter data.

### RELATED APPLICATIONS

This application claims the benefit of the filing date pursuant to 35 U.S.C. § 120 of Application Serial No. 60/413,392, for a HOSPITAL BED WITH INTEGRATED DISPLAY, filed Sep. 25, 2002, the disclosure and content of which is hereby incorporated by reference in its, entirety.

### FIELD OF THE INVENTION

The present invention relates to a patient monitoring system for providing increased acuity flexibility, the system including integrated vital signs monitoring capability and, more particularly, a bed with an integrated display for receiving and displaying a patient's medical data such as medical history, laboratory test results, x-rays or other radiological data, medication or other pharmacy data; and/or physiological data, such as EKG signals, blood pressure data, temperature readings, pulse, respiration rate data, pulse oximeter data, entertidal CO2 concentrations, cardiac output, pulmonary artery pressures, peripheral vascular resistance, oxygen consumption, and oxygen delivery to tissues.

### BACKGROUND OF THE INVENTION

The present invention relates to a patient care system with an integrated display for receiving and displaying a patient's physiological and/or medical data. Typically, a patient's vital signs or physiological data is measured or derived by electronic measurement systems. Conventional measurement systems utilize various detectors connected to the patient for detecting the patient's physiological data. In addition, those measurement systems utilize cables or wires to transmit the physiological data from the detectors to a display, monitor, or other electronic equipment located adjacent that patient's hospital bed.

With the advent of shorter in-patient hospital stays per patient, hospitalized patients are more acutely ill than in the past and require greater monitoring to provide optimal care. The wires leading to the various displays, monitors, and other equipment, however, are cumbersome and uncomfortable for the patient. Moreover, the displays, monitors, and other equipment utilized by those systems clutter up the area surrounding the hospital bed, immobilize the patient and potentially increases the number of hospital acquired infections transmitted by reuse of wires. Consequently, the numerous wires, displays, monitors, and other equipment hinder the patient's mobility and make it difficult for health care providers to move around the hospital bed while administering medical care to the patient. Thus, a need exists for an integrated patient care system that eliminates the cumbersome wires, monitors, displays, and other equipment utilized by conventional measurement systems, and provides increased acuity flexibility.

### BRIEF DESCRIPTION OF THE DRAWING

The foregoing aspects and many of the advantages of the present invention will become readily appreciated by reference to the following detailed description of the preferred embodiment, when taken in conjunction with the accompanying drawings, wherein:

FIG. 1 depicts an exemplary embodiment of the hospital bed with an integrated display in a docked position;

FIG. 2 depicts an exemplary embodiment of the hospital bed with an integrated display in the undocked position;

FIG. 3 depicts an exemplary embodiment of the hospital bed with an integrated display in the swiveled position;

FIG. 4 depicts a flow chart showing possible data pathways from patient and/or patient related services; and

FIG. 5 depicts an exemplary embodiment of the remote electronics unit utilized with the present invention.

### DETAILED DESCRIPTION OF THE INVENTION

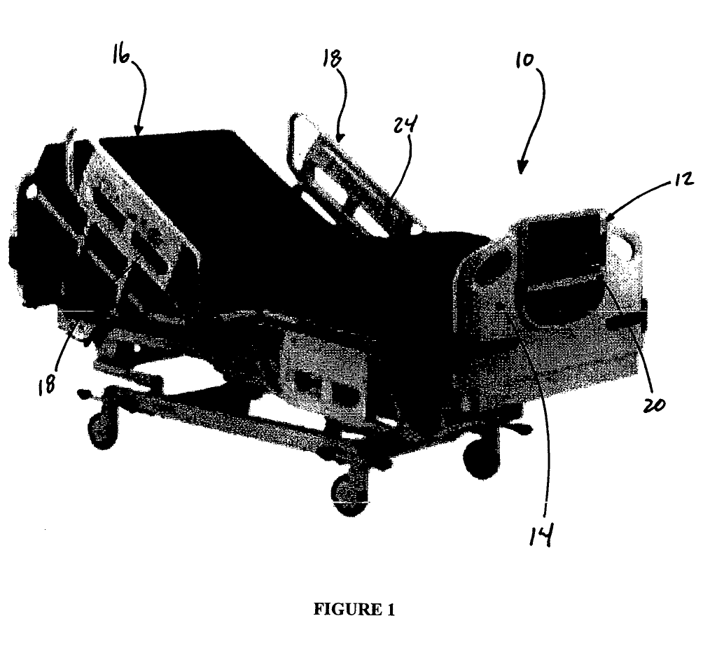

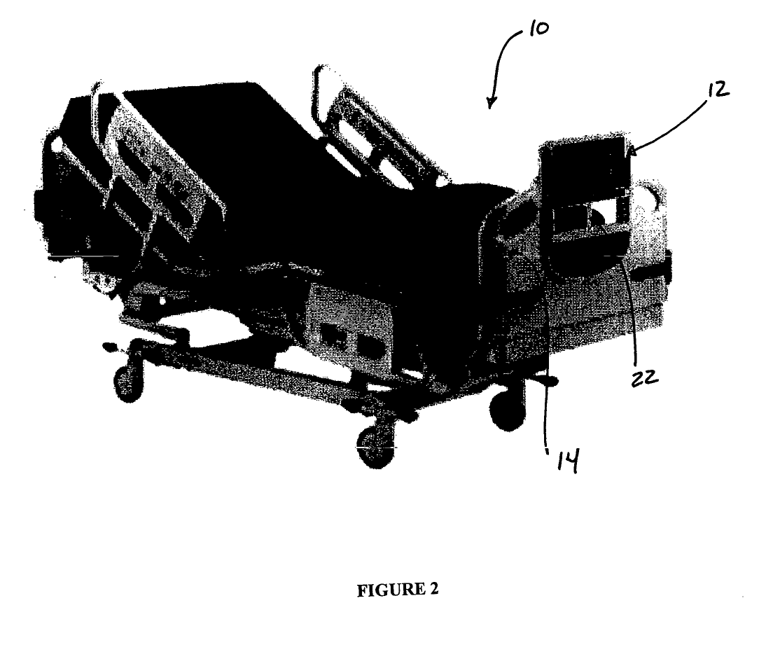

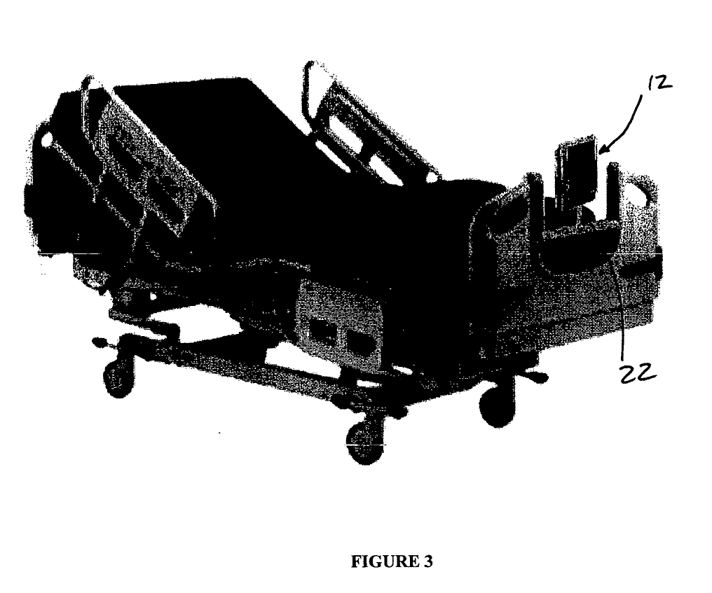

For a better understanding of the present invention, reference may be had to the following detailed description taken in conjunction with the accompanying drawings. FIG. 1 depicts a hospital bed 10 with an integrated patient display 12 for receiving and displaying a patient's physiological data and/ or medical records or other data. Such physiological data may include EKG signals, blood pressure data, temperature readings, pulse, respiration rate data, pulse oximeter data, entertidal CO2 concentrations, cardiac output, pulmonary artery pressures, peripheral vascular resistance, oxygen consumption, and oxygen delivery to tissues. Medical records data may include patient charts, laboratory results, radiological data, current and historical medications, or other relevant patient data. In the preferred embodiment, the patient display 12 is located on a footboard 14 of the patient bed 10. The patient display 12, however, may be located anywhere on the hospital bed 10 without departing from the spirit and scope of the invention. For example, other possible locations include the headboard 16 or the bed rails 18. As shown in FIG. 1, the patient display 12 is located in a display bay 20, which is recessed into the footboard 14. FIG. 1 depicts the patient display 12 in the docked position. As shown in FIG. 2, the patient display 12 is slidably connected to the footboard 14 via a swivel arm 22. The patient display 12 can be moved from the docked position to an undocked position by slidably moving the patient display 12 up the swivel arm 22. As shown in FIG. 3, the patient display 12 and swivel arm 22 may be constructed such that the patient display 12 can rotate or swivel about a vertical axis defined by the swivel arm 22. Preferably, the patient display 12 is capable of 360° rotation about the swivel arm 22. Thus, a health care provider can position the display such that the display can be viewed from any location in the room.

The patient display 12 may be powered by a battery and/or a wired connection. The battery may be configured to insert into a battery port located on the patient display 12. The battery may be retained in the battery port by latches or other suitable fastening means, such as clips, screws or the like. The battery may be a 3.6 V Li-ion rechargeable battery. The battery may also have a wired connection for plugging into an electrical connection on the hospital bed or any standard wall socket for recharging.

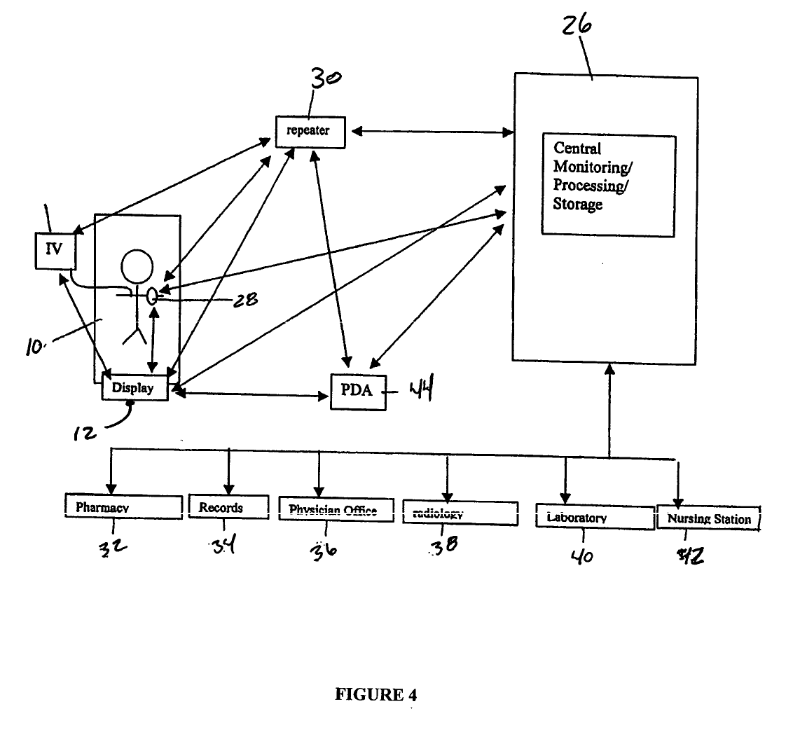

The patient's physiological date is collected from the physiological detectors that are connected to the patient. Referring back to FIG. 1, the hospital bed may include a detector interface 24 for connecting the physiological detectors (not shown) to the hospital bed 10. The physiological data is transferred from the detectors to the hospital bed 10 via wires or cable, which are connected to the detector interface 24. The physiological data is then transmitted to a physiological data processor (not shown) that processes the physiological data before it is displayed on the patient display 12. The physiological data processor may either be integrated into the hospital bed 10 or integrated into the patient display 12. As shown in FIG. 4, the physiological data can also be transmitted to a remotely located central monitoring/processing station 26 that processes and/or records the physiological data. This central monitoring/processing station 26 can also forward the processed data and/or other recorded data to other locations inside the hospital or outside the hospital (as discussed in more detail below). In addition, the physiological data may be viewed at the central monitoring/processing station 26. Thus, health care providers that are located both outside and inside the patient's hospital room may view the patient's physiological data.

Communication between the central monitoring/processing station 26 and the physiological data processor, which may be integrated into the hospital bed 10 or patient monitor 12, may take place via a wired connection, such as a hospital network. Alternatively, communication between the physiological data processor and the central monitoring/processing station 26 may take place via a wireless connection, such as a telemetry system or the like.

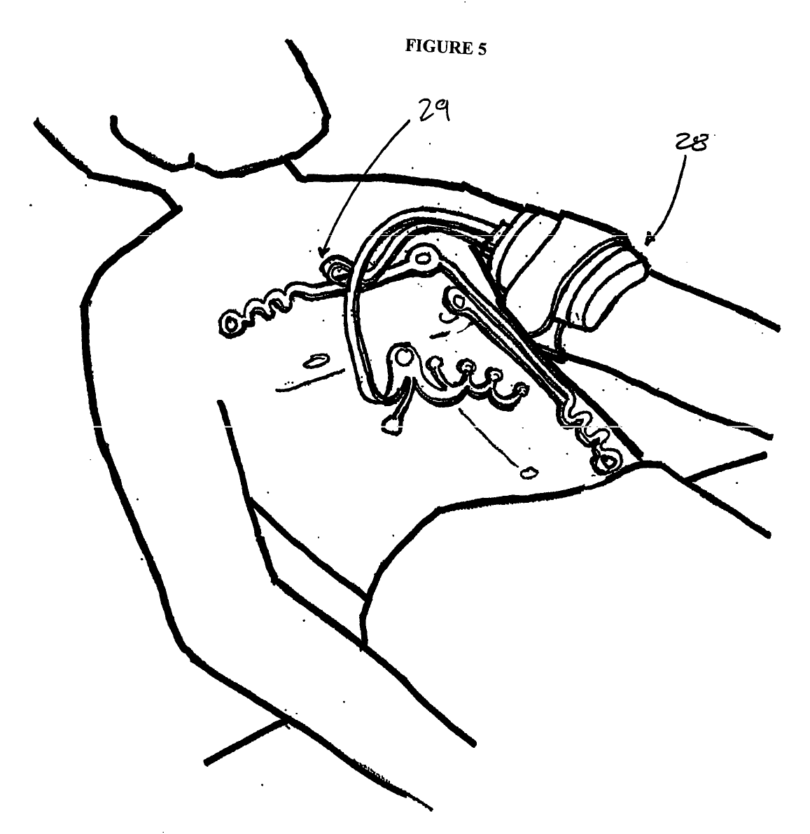

In an exemplary embodiment of the present invention, the patient's physiological data may be wirelessly transmitted to the patient display 12 by a remote electronics unit 28. One such embodiment is shown in FIG. 5. The physiological detector 29 connects to the patient and the physiological data is transmitted from the patient to the remote electronics unit 28. The physiological detector 29 may comprise a chest assembly and/or a precordial assembly. An exemplary physiological detector 29 and remote electronics unit 28 are described in co-pending U.S. patent application Ser. No. 09/998,733, entitled “WIRELESS ECG SYSTEM” (Attorney Docket No. 005123.0004) filed on Nov. 30, 2001, the content of which is incorporated herein by reference in its entirety.

The remote electronics unit 28 may be positioned on the patient, as shown in FIG. 5, or be removably attached to the hospital bed 10 (not shown). The remote electronics unit 28 wirelessly transmits the physiological data to a receiver (not shown) connected to a processor (not shown). The receiver receives the physiological data from the remote electronics and the processor processes the physiological data before it is displayed on the patient display 12. The receiver and processor may be either integrated into the patient display 12 or integrated into the hospital bed 10. Alternatively, the receiver and processor may be integrated into a base station as described in co-pending U.S. patent application Ser. No. 09/998,733. The base station may have a plurality of terminals for connecting to the patient display 12.

In addition, as depicted in FIG. 4, the remote electronics unit 28 may transmit the physiological data to the central monitoring/processing station 26. The remote electronics unit 28 may transmit the data directly to the remote viewing station or indirectly via a telemetry system. A wireless version of such a telemetry system is described in co-pending U.S. patent application Ser. No. 10/349,486, entitled “ECG TELEMETRY SYSTEM” (which claims the benefit of the filing date pursuant to 35 U.S.C. § 120 of Application Serial No. No. 60/350,840, filed on Jan. 22, 2002), filed on Jan. 22, 2003, the content of which is incorporated herein by reference in its entirety.

When a telemetry system as disclosed in U.S. patent application Ser. Nos. 10/349,486 is utilized, the remote electronics unit 28 transmits the physiological data to a repeater 30. The repeater 30 is capable of receiving and relaying data transmissions from multiple remote electronics units 28 simultaneously. Multiple repeaters 30 are positioned in locations throughout the hospital to provide cell pattern coverage consisting of overlapping zones so that each patient using the system will be within the range of multiple repeaters 30 at any given time. Each repeater 30, in turn, relays the transmissions from the remote electronics unit 28 to the central monitoring/processing station 26. The central monitoring/processing station 26 will process the data transmissions and transmit the processed data to the patient display 12. The central monitoring/processing station 26 can be configured to transmit the processed data to the patient display 12 via wireless and/or wired communication. In an alternate implementation, the repeater 30 could provide zone coverage using one or more antennas positioned in spaced relationship to each other.

In addition, the receiver and processor, which may be either integrated into the patient display 12, the hospital bed 10, or the base station, may be capable of receiving data from the central monitoring/processing station 26. In addition, the remote electronics unit 28 may also be capable of receiving data from the central monitoring/processing station 26. In other words, the receiver, processor and/or remote electronics unit 28 may be capable of two-way communication with the central monitoring/processing station. In addition, the receiver and processor can be interfaced with an electronic medical records system or software and used to display comprehensive data pertaining to the patient on the patient display 12. Such data may include, for example, patient charts, historical medical records, laboratory test results, radiology results, current and historical medications and other pharmacy data or any other patient linked data. Suitable medical records software is being developed and marketed by a number of companies, including Omnichart® from Medical Manager Health Systems. The patient display 12 may optionally include a data entry means or device, such as a touch screen, handwriting recognition, keyboard, bar code reader or voice recognition means to facilitate data entry into the medical records system or software. Data entered by a health care provider would be retained and/or processed by the electronic medical records system or software. Such data could include any desired information including but not limited to revised medications, nursing orders, dietary requirements and/or patient tests.

In yet another embodiment, as shown in FIG. 4, the remote electronics unit 22 may transmit the unprocessed physiological data to the remote viewing central monitoring/processing station 26 without transmitting the data to the patient display 12. In such an embodiment, the unprocessed physiological data is processed at the central monitoring/processing station 26. A transmitter (not shown) at the central monitoring/processing station 26 wirelessly transmits the processed data to the patient display 12 where the data is displayed. The processed physiological data may optionally be transmitted and/or displayed in a pharmacy 32, a medical records department 34, a physicians office 36, a radiology department 38, a laboratory 40, a nursing station 42, and/or a personal digital assistant (PDA) 44 or other local display, and/or any other location inside or outside the hospital where the patient data is desired.

In another embodiment, the patient display 12 is wireless and portable and may be temporarily affixed to the bed, wall or any other convenient piece of furniture or fixture. For example, the patient display 12 may be portable and capable of attaching to a wheel chair, pole, or other suitable mobile transport mechanism or device. The patient display 12 may be attached to any suitable mobile transport mechanism via a wide variety of attachment methods, such as clips, screws, magnets, tongue and groove attachment, or the like. Further, the patient display 12 could be attached to or placed on any suitable wall unit or fixture, which are typically located throughout the hospital room or ward and are commonly used to hold medical equipment.

In addition, the patient display 12 may take the form of a personal digital assistant (PDA) and/or a notebook or tablet type computer carried by a health care provider.

The physiological data could be received by the patient display 12 as described above in this application. Such a patient display 12 would have the advantage of making any hospital bed within reach of a wired or wireless network a monitored bed by placing the patient display 12 in the patient room.

In another embodiment, the receiver and processor, which may be either integrated capable of receiving data pertaining to multiple patients. Such data may come from other patient displays 12, the central monitoring/processing station 26, and/or any other location from the hospital. When a patient display 12 is capable of receiving data from more than one patient, a means for securing the data and/or confirming which data pertains to which patient may be necessary. Use of barcode readers, token/key systems, radio frequency tags, and other means of pairing that are known in the art may be used to pair the information to various patients. In such embodiment, the patient display 12 will be capable of simultaneously displaying the physiological data and/or medical records from multiple patients. Thus, a health care provider will be able to simultaneously monitor the physiological data and/or access the medical records of multiple patients.

In the foregoing specification, the present invention has been described with reference to specific exemplary embodiments thereof. It will be apparent to those skilled in the art, that a person understanding this invention may conceive of changes or other embodiments or variations, which utilize the principles of this invention without departing from the broader spirit and scope of the invention. The specification and drawings are, therefore, to be regarded in an illustrative rather than restrictive sense.

We claim:

1. A system for monitoring data comprising:
a remote electronics unit for acquiring physiological data from a patient and for wirelessly transmitting the physiological data to a base station, the base station comprising a plurality of terminals for transmitting the physiological data to a patient display.

2. The system of claim 1 wherein the patient display is configured to be attached to a hospital bed.

3. The system of claim 2 wherein the patient display is configured to be rotatably connected to a swivel arm.

4. The system of claim 3 wherein the patient display is capable of rotating about a vertical axis defined by the swivel arm.

5. The system of claim 1 wherein the patient display is configured to be attached to a transport mechanism.

6. The system of claim 1 wherein the patient display is configured to be connected to a wall unit.

7. The system of claim 1 further comprising a chest assembly configured to attach to the remote electronics unit for collecting the physiological data from the patient and transmitting the physiological data to the remote electronics unit.

8. The system of claim 1 wherein the physiological data pertains to information selected from the group consisting EKG signals, blood pressure data, temperature readings, pulse, respiration rate data, pulse oximeter data, entertidal CO2 concentrations, cardiac output, pulmonary artery pressures, peripheral vascular resistance, oxygen consumption, and oxygen delivery to tissues.

9. The system of claim 1 wherein the patient display is configured to be displayed the patient's medical records.

10. The system of claim 1 further comprising a central monitoring station for receiving the physiological data from the patient.

11. The system of claim 10 wherein the remote electronics unit is configured to wirelessly transmit the physiological data to the central monitoring station.

12. The system of claim 10 wherein the central monitoring station is configured to receive the physiological data via wired transmission.

13. The system of claim 10 wherein the central monitoring station is capable of two-way communication with the remote electronics unit.

14. The system of claim 10 wherein the central monitoring station is capable of two-way communication with the patient display.

15. The system of claim 1 wherein the patient display comprises a data entry device.

16. A system for monitoring the physiological data associated with at least one patient comprising, in combination:
at least one remote electronics unit removably connected to a chest assembly for acquiring physiological signals from a patient, the remote electronics unit comprising a transmitter for transmitting the physiological signals;
at least one repeater comprising a receiver for receiving the physiological signals from the body electronics unit and a transmitter for transmitting the physiological signals;
a central monitoring station comprising a receiver for wirelessly receiving the physiological signals from the at least one repeater;
at least one patient display for receiving the physiological signals from the central monitoring station.

17. The system of claim 16 wherein the at least one patient display is configured to be attached to a hospital bed.

18. The system of claim 17 wherein the at least one patient display is configured to be rotatably connected to a swivel arm.

19. The system of claim 18 wherein the at least one patient display is capable of rotating about a vertical axis defined by the swivel arm.

20. The system of claim 16 wherein the at least one patient display is configured to be attached to a transport mechanism.

21. The system of claim 16 wherein the at least one patient display is configured to be connected to a wall unit.

22. The system of claim 16 wherein the physiological data pertains to information selected from the group consisting EKG signals, blood pressure data, temperature readings, pulse, respiration rate data, pulse oximeter data, entertidal CO2 concentrations, cardiac output, pulmonary artery pressures, peripheral vascular resistance, oxygen consumption, and oxygen delivery to tissues.

23. The system of claim 16 wherein the at least one patient display is capable of displaying the patient's medical records.

24. The system of claim 16 wherein the central monitoring station is capable of two-way communication with the at least one remote electronics unit.

27. The system of claim 16 wherein the central monitoring station is capable of two-way communication with the patient display.

28. The system of claim 16 wherein the patient display comprises a data entry device.

29. The system of claim 16 further comprising an electronic medical records system.

30. A system for monitoring data comprising:
a remote electronics unit for acquiring physiological data from a patient;
a central monitoring station for receiving the physiological signals from the remote electronics unit, the central monitoring station comprising a processor for processing the physiological signals and a transmitter for transmitting the processed signals;
a patient display for receiving the processed physiological signals from the central monitoring station, the patient display is configured to be attached to a hospital bed.

31. The system of claim 30 wherein the patient display is configured to be rotatably connected to a swivel arm.

32. The system of claim 31 wherein the patient display is capable of rotating about a vertical axis defined by the swivel arm.

33. The system of claim 29 wherein the patient display is configured to be attached to a transport mechanism.

34. The system of claim 29 wherein the patient display is configured to be connected to a wall unit.

35. The system of claim 29 further comprising a chest assembly configured to attach to the remote electronics unit for collecting the physiological data from the patient and transmitting the physiological data to the remote electronics unit.

36. The system of claim 29 wherein the physiological data pertains to information selected from the group consisting EKG signals, blood pressure data, temperature readings, pulse, respiration rate data, pulse oximeter data, entertidal CO2 concentrations, cardiac output, pulmonary artery pressures, peripheral vascular resistance, oxygen consumption, and oxygen delivery to tissues.

37. The system of claim 29 wherein the patient display is capable of displaying the patient's medical records.

38. The system of claim 29 wherein the central monitoring station is capable of two-way communication with the remote electronics unit.

39. The system of claim 29 wherein the central monitoring station is capable of two-way communication with the patient display.

40. The system of claim 29 wherein the patient display comprises a data entry device.

41. The system of claim 1 further comprising an electronic medical records system.
